(19) United States
(12) Patent Application Publication
(10) Pub. No.: US 2026/0262990 A1
(43) Pub. Date: Sep. 10, 2026

(54) Wearable Device and Method of Cardiovascular Monitoring

(71) Applicant: Huawei Technologies Co., Ltd., Shenzhen (CN)

(72) Inventors: Virginie Vissac, Munich (DE); Heikki Vilho Nieminen, Helsinki (FI)

(21) Appl. No.: 18/721,973

(22) PCT Filed: Dec. 21, 2021
(86) PCT No.: PCT/EP2021/086966

§ 371 Date: Jun. 20, 2024

(51) Int. Cl. A61B 5/318; A61B 5/00; A61B 5/0205; A61B 5/021; A61B 5/024; A61B 5/1455

(52) U.S. Cl. CPC A61B 5/318; A61B 5/0205; A61B 5/14551; A61B 5/681; A61B 5/02125; A61B 5/02416; A61B 5/02438; A61B 2562/04; A61B 2562/06

(57) ABSTRACT

A wearable device includes a housing, a first electrode, a second electrode, a first pulse oximeter, a second pulse oximeter, a processing module, and an output module. The housing is configured to be placed with a lower face against a user's skin on a first arm. The first electrode is arranged on the lower face. The second electrode is arranged on a different face of the housing. The first pulse oximeter is arranged on the lower face and configured to output a first photoplethysmogram (PPG) signal when the lower face is placed against the user's skin. The second pulse oximeter is configured to output a second PPG signal when a second arm of the user is placed in contact with the second electrode and the second pulse oximeter.

### TECHNICAL FIELD

The present disclosure relates generally to the field of wearable devices used for health assessment of a user; and more specifically, to an electronic device to be worn on the arm, wrist or finger of a user and a method for cardiovascular risk assessment of the user wearing the electronic device.

### BACKGROUND

Cardiovascular monitoring is a process of continuous or intermittent monitoring of heart activity, aiming at detecting cardiovascular diseases. The cardiovascular diseases are the major cause of death worldwide, accounting for 31% of total death happening in the world according to the World Health Organization (WHO). Cardiovascular diseases are often caused by a poor diet and a lack of exercise. Moreover, they generally go unnoticed because most examinations targeting the heart are done in medical facilities and in hospitals with a high range of equipment where the patient will go only after having experienced the first symptoms. If the cardiovascular diseases could be detected at an earlier stage, the patients could change their way of life to improve their cardiovascular health before the first symptoms are experienced and medication is required.

One of the major cardiovascular diseases is atherosclerosis, which causes plaque (i.e. made of fat, cholesterol, calcium, or other substances that can be found in the blood) to build up in arteries. With time, the plaque hardens and narrows the arteries which will cause a limitation of oxygen-rich blood flow to organs and parts of the body placed after the blockage. Different diseases are associated with atherosclerosis depending on concerned arteries, coronary heart disease when the plaque builds up in the coronary arteries limiting the blood supply to the heart, a carotid artery disease, CAD, when it affects the carotid arteries limiting the blood supply to the brain, a peripheral artery disease, PAD, when it affects the arteries supplying arms, legs or pelvis and chronic kidney disease when this affects renal arteries. Apart from the fact that it may reduce blood supply, atherosclerosis also presents the risk of thrombosis or embolism.

Most of the time, there will be no symptoms experienced by the patients until a blockage has occurred. As atherosclerosis is mainly asymptomatic, it is hard to determine its incidence however it is considered as a major cause of cardiovascular disease. For example, 75% of acute myocardial infarctions are caused by plaque rupture. According to a study published in The Lancet in 2020, the prevalence of carotid artery disease is 1.5% worldwide with 21.1% of the population presenting carotid plaque. Atherosclerosis also accounts for 90% of the cases of PAD which concerns 200 million people. Diagnosing atherosclerosis in current methods includes blood tests, electrocardiogram, ECG, chest X-ray, echocardiography, computed tomography scan, stress testing, angiography, and ankle-brachial index. But these methods need to be done by medical professionals in specific facilities where the patient will only go if he has already been diagnosed as being at risk or is experiencing some symptoms.

Existing solutions to detect diseases caused by atherosclerosis include ankle-brachial index and listening to turbulence sounds with a stethoscope for PAD and CAD respectively. However even for these simpler methods there is a need for a specialist or a medical professional to provide a specific consultation to the patients. Other medical systems using tonometric probes to measure the carotid-femoral pulse wave velocity and aiming at measuring a stiffening of the arteries are bulky and difficult to use in addition to leaving the peripheral arteries out of the examination. Other easier but less precise solutions embedded into wearable devices are generally only looking at one of the arteries i.e. one arm or one leg, to deduce information. Atherosclerosis however generally presents an asymmetrical development and therefore can go unnoticed if it is mainly present in a non-observed limb i.e. another arm or another leg. Furthermore, such devices aiming at determining the blood pressure non-invasively without a cuff using pulse wave velocity method generally focus on the wrist, however the radial artery has a property to change its radius to adapt to the blood flow therefore making the link between pulse wave velocity and blood pressure not unique (as it changes with the radius of the artery). As a consequence, when measuring the pulse wave velocity on the radial artery with no other information it becomes difficult to calculate the corresponding blood pressure.

Therefore, the present invention aims to provide a wearable device of existing systems or technologies to determine at an early stage the risk of a user of having or developing a cardiovascular disease.

### SUMMARY

It is an object of the disclosure to provide a wearable device for accurate calculation of a cardiovascular health of a user, and a method of cardiovascular monitoring for accurate calculation of the cardiovascular health of the user while avoiding one or more disadvantages of prior art approaches.

This object is achieved by the features of the independent claims. Further implementations are apparent from the dependent claims, the description, and the figures.

The disclosure provides a wearable device and a method of cardiovascular monitoring.

According to a first aspect, there is provided a wearable device. The wearable device includes a housing, a first electrode, a second electrode, a first pulse oximeter, a second pulse oximeter, a processing module and an output module. The housing is configured to be placed with a lower face against a user's skin on a first arm. The first electrode is arranged on the lower face of the housing, a second electrode arranged on a different face of the housing. The first pulse oximeter is arranged on the lower face of the housing and configured to output a first photoplethysmogram, PPG, signal when the lower face is placed against the user's skin. The second pulse oximeter is arranged on a different face of the housing and configured to output a second PPG signal when a second arm of the user is placed in contact with the second electrode and the second pulse oximeter. The processing module is configured to (i) measure an ECG signal using the first electrode and the second electrode when the housing is placed with a lower face against the user's skin on the first arm and the second arm of the user is placed in contact with the second electrode, (ii) measure a pulse arrival time, PAT, for each arm based on the respective PPG signals and the ECG signal, (iii) measure a pulse transit time, PTT, based on the difference between the measured PATs, and (iv) determine whether a pulse wave velocity, PWV, for each arm is equal or not equal, based on the measured PATs and PTT and an offset between an expected location of the device along the first arm and an expected contact point along the second arm and/or a pulse waveform analysis of the two PPG signals. The output module is configured to generate an output in response to determining that the PWV for each arm is not equal.

The wearable device provides calculation of a risk of having or developing a Peripheral Artery Disease, PAD, or a Carotid Artery Disease, CAD. The wearable device may be worn on any of arms, wrists, or fingers of the user and studies blood flow in both of the user's arms to determine a cardiovascular risk. The wearable device calculates the risk of having or developing a PAD or a CAD by evaluating the symmetry of parameters measured in both arms. The wearable device includes a combination of measurements to obtain an indication of a cardiovascular health of the user and to improve a calculation of aortic pulse wave velocity, PWV. The wearable device evaluates the cardiovascular health of the user by calculating peripheral PWV in the arms and including pulse arrival times, PATs to approximate an aortic PWV for the user with a symmetrical PWV in both arms. The wearable device determines a blood pressure of the user using the aortic PWV.

Optionally, the processing module is further configured to: calculate oxygen saturation values and pulse rise times, PRTs, for the first arm and the second arm, based on a combination of a plurality of PPG signals from the respective pulse oximeter on the respective arm, and compare the SPO2 values and PRTs for the first arm and the second arm to identify a potential obstruction. Optionally, the output module is further configured to generate an output in response to any potential obstruction.

Optionally, in response to determining that the PWV for each arm is equal, the processing module is further configured to calculate a peripheral PWV based on the measured PATs and PTT. Optionally, the output module is further configured to generate an output in response to a value of the peripheral PWV above a predefined threshold.

Optionally, the processing module is further configured to: calculate an aortic PWV from the calculated peripheral PWV and the measured PATs. Optionally, the output module is further configured to generate an output in response to a value of the aortic PWV outside a predefined range.

Optionally, the processing module is further configured to calculate a blood pressure of the user based on the calculated aortic PWV. Optionally, the calculation is calibrated using an external blood pressure measurement. Optionally, the output module is further configured to generate an output based on the calculated blood pressure.

Optionally, the output module includes a screen and/or speaker and/or transmission unit.

Optionally, the wearable device further includes a third electrode arranged on the lower face of the housing. Optionally, the third electrode is connected to ground and the processing module is configured to measure the ECG signal additionally using the third electrode.

Optionally, each pulse oximeter is configured to use one or more wavelengths to generate a corresponding number of PPG signals.

Optionally, each pulse oximeter includes at least a red, infrared and green LED.

Optionally, one or both of the second electrode and the second pulse oximeter are integrated with a button of the wearable device.

Optionally, the first pulse oximeter is configured to operate in a reflection mode.

Optionally, the housing is formed to encircle at least a portion of the first arm and the first pulse oximeter including two elements configured to operate in a transmission mode.

Optionally, the offset and/or one or more biometric lengths of the user are estimated using one or more parameters of the user and/or measured using a predefined calibration step.

According to a second aspect, there is provided a method of cardiovascular monitoring. The method includes placing a wearable device with a lower face against a user's skin on a first arm. The wearable device includes a first electrode arranged on the lower face of the housing, a second electrode arranged on a different face of the housing, a first pulse oximeter arranged on the lower face of the housing and configured to output a first photoplethysmogram, PPG, signal, and a second pulse oximeter arranged on a different face of the housing and configured to output a second PPG signal. The method includes placing a second arm of the user in contact with the second electrode and the second pulse oximeter, measuring an ECG signal using the first electrode and the second electrode, measuring a pulse arrival time, PAT, for each arm based on the respective PPG signals and the ECG signal, measuring a pulse transit time, PTT, based on the difference between the measured PATs and caused by an offset between an expected location of the wearable device along the first arm and an expected contact point along the second arm, determining whether a pulse wave velocity, PWV, for each arm is equal or not equal, based on the measured PATs and PTT and information of the user such as height and gender and/or pulse wave analysis of the PPG signals acquired, and generating an output in response to determining that the PWV for each arm is not equal.

The method provides calculation of a risk of having or developing a Peripheral Artery Disease, PAD, or a Carotid Artery Disease, CAD. The method calculates the risk of having or developing a PAD or a CAD by evaluating the symmetry of parameters measured in both arms. The method includes a combination of measurements to obtain an indication of a cardiovascular health of the user and to improve a calculation of aortic pulse wave velocity, PWV. The method evaluates the cardiovascular health of the user by calculating a peripheral PWV in the arms and including pulse arrival times, PATs to approximate an aortic PWV for the user with a symmetrical PWV in both arms. The method determines a blood pressure of the user using the aortic PWV.

Optionally, the method includes calculating, based on the first pulse oximeter and the second pulse oximeter signals, oxygen saturation values and pulse rise times, PRTs, for the first arm and the second arm. The method includes comparing the SPO2 values and PRTs for the first arm and the second arm to identify a potential obstruction, and generating an output in response to any potential obstruction.

Optionally, the method further includes if the PWV for each is equal, calculating a peripheral PWV based on the measured PATs and PTT, and generating an output in response to a value of the peripheral PWV above a predefined threshold.

Optionally, the method further includes calculating an aortic PWV from the calculated peripheral PWV and the measured PATs, and generating an output in response to a value of the aortic PWV outside a predefined range.

Optionally, the method further includes calculating a blood pressure of the user based on the calculated aortic PWV. Optionally, the calculation is calibrated using an external blood pressure measurement. The method further includes generating an output based on the calculated blood pressure.

According to a third aspect, there is provided a computer-readable medium including instructions which, when executed by a processor, cause the processor to perform the method.

Therefore, in contradistinction to the prior art, according to the wearable device and the method, provides an accurate calculation of a cardiovascular risk of a user. The wearable device and the method improve the calculation of the aortic PWV to obtain accurate indications on the cardiovascular health of the user.

These and other aspects of the disclosure will be apparent from the implementations described below.

### BRIEF DESCRIPTION OF DRAWINGS

Implementations of the disclosure will now be described, by way of example only, with reference to the accompanying drawings, in which:

FIG. 1 is a block diagram of a wearable device in accordance with an implementation of the disclosure;

FIGS. 2A-2B illustrate exemplary diagrams of a front side and a back side of a wearable device in accordance with an implementation of the disclosure;

FIGS. 3A-3F illustrate exemplary diagrams of a wearable device in accordance with an implementation of the disclosure;

FIG. 4 depicts a graphical representation of one or more signals acquired by a wearable device in accordance with an implementation of the disclosure;

FIG. 5 illustrates a flow diagram of a sequence of measurement and acquisition of parameters in a wearable device in accordance with an implementation of the disclosure;

FIG. 6 illustrates a schematic diagram of an algorithm to determine a cardiovascular risk of a user in accordance with an implementation of the disclosure; and

FIGS. 7A-7C are flow diagrams that illustrate a method of cardiovascular monitoring in accordance with an implementation of the disclosure.

### DETAILED DESCRIPTION OF THE DRAWINGS

Implementations of the disclosure provide a wearable device for accurate calculation of a cardiovascular health of a user, and a method of cardiovascular monitoring for accurate calculation of the cardiovascular health of the user.

To make solutions of the disclosure more comprehensible for a person skilled in the art, the following implementations of the disclosure are described with reference to the accompanying drawings.

Terms such as “a first”, “a second”, “a third”, and “a fourth” (if any) in the summary, claims, and foregoing accompanying drawings of the disclosure are used to distinguish between similar objects and are not necessarily used to describe a specific sequence or order. It should be understood that the terms so used are interchangeable under appropriate circumstances, so that the implementations of the disclosure described herein are, for example, capable of being implemented in sequences other than the sequences illustrated or described herein. Furthermore, the terms “include” and “have” and any variations thereof, are intended to cover a non-exclusive inclusion. For example, a process, a method, a system, a product, or a device that includes a series of steps or units, is not necessarily limited to expressly listed steps or units but may include other steps or units that are not expressly listed or that are inherent to such process, method, product, or device.

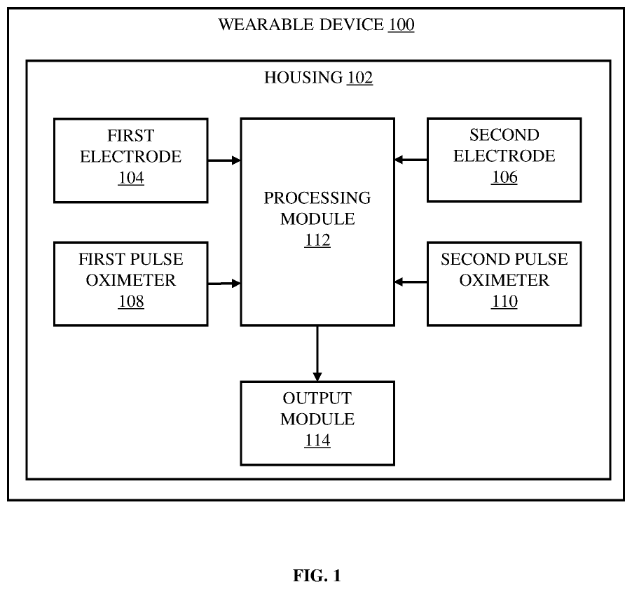

FIG. 1 is a block diagram of a wearable device 100 in accordance with an implementation of the disclosure. The wearable device 100 includes a housing 102, a first electrode 104, a second electrode 106, a first pulse oximeter 108, a second pulse oximeter 110, a processing module 112, and an output module 114. The housing 102 is configured to be placed with a lower face against a user's skin on a first arm. The first electrode 104 is arranged on the lower face of the housing 102. The second electrode 106 is arranged on a different face of the housing 102. The first pulse oximeter 108 is arranged on the lower face of the housing 102 and configured to output a first photoplethysmogram, PPG, signal when the lower face is placed against the user's skin. The second pulse oximeter 110 is arranged on a different face of the housing 102 and configured to output a second PPG signal when a second arm of the user is placed in contact with the second electrode 106 and the second pulse oximeter 110. The processing module 112 is configured to (i) measure an ECG signal using the first electrode 104 and the second electrode 106 when the housing 102 is placed with a lower face against the user's skin on the first arm and the second arm of the user is placed in contact with the second electrode 106, (ii) measure a pulse arrival time, PAT, for each arm based on the respective PPG signals and the ECG signal, (iii) measure a pulse transit time, PTT, based on the difference between the measured PATs, and (iv) determine whether a pulse wave velocity, PWV, for each arm is equal or not equal, based on the measured PATs and PTT and an offset between an expected location of the device along the first arm and an expected contact point along the second arm and/or a pulse waveform analysis of the two PPG signals. The output module 114 is configured to generate an output in response to determining that the PWV for each arm is not equal.

The wearable device 100 provides a calculation of a risk of having or developing a cardiovascular disease and in particular Peripheral Artery Disease, PAD and Carotid Artery Disease, CAD. The wearable device 100 may be worn on any of arms, wrists, or fingers of the user. The wearable device 100 calculates the risk of having or developing a PAD or a CAD by evaluating the symmetry of parameters measured in both arms. The wearable device 100 includes a combination of measurements to obtain an indication of a cardiovascular health of the user and to improve a calculation of aortic pulse wave velocity, PWV. The wearable device 100 evaluates the cardiovascular health of the user by calculating peripheral PWV in the arms and including pulse arrival times, PATs to approximate an aortic PWV for the user with a symmetrical PWV in both arms. The wearable device 100 determines a blood pressure of the user using the aortic PWV.

The wearable device 100 may enable checking of symmetry between the arms that relates to a cardiovascular risk. The wearable device 100 evaluating the symmetry with parameters including oxygen saturation, pulse rise time, or pulse wave velocity. The wearable device 100 includes the first electrode 104 and the second electrode 106 for the ECG measurement between the two arms of the user, and one or more SPO2 sensors. The one or more SPO2 sensors may include a first SPO2 sensor that is in contact with the arm, the wrist, or the finger wearing the wearable device 100 of the user and a second SPO2 sensor that is in contact with a part of the opposite hand of the user. Optionally, the one or more SPO2 sensors include at least a red, infrared, and green light-emitting diodes, LEDS.

Optionally, the processing module 112 is further configured to: calculate oxygen saturation values and pulse rise times, PRTs, for the first arm and the second arm, based on a combination of a plurality of PPG signals from the respective pulse oximeters on the respective arms, and compare the SPO2 values and PRTs for the first arm and the second arm to identify a potential obstruction. Optionally, the output module 114 is further configured to generate an output in response to any potential obstruction.

Optionally, in response to determining that the PWV for each arm is equal, the processing module 112 is further configured to calculate a peripheral PWV based on the measured PATs and PTT. Optionally, the output module 114 is further configured to generate an output in response to a value of the peripheral PWV above a predefined threshold.

Optionally, the processing module 112 is further configured to: calculate an aortic PWV from the calculated peripheral PWV and the measured PATs. Optionally, the output module 114 is further configured to generate an output in response to a value of the aortic PWV outside a predefined range.

Optionally, the processing module 112 is further configured to calculate a blood pressure of the user based on the calculated aortic PWV. Optionally, the calculation is calibrated using an external blood pressure measurement. Optionally, the output module 114 is further configured to generate an output based on the calculated blood pressure.

Optionally, the output module 114 includes a screen and/or speaker and/or transmission unit.

Optionally, the wearable device further includes a third electrode arranged on the lower face of the housing 102. Optionally, the third electrode is connected to ground and the processing module 112 is configured to measure the ECG signal additionally using the third electrode.

Optionally, each pulse oximeter is configured to use one or more wavelengths to generate a corresponding number of PPG signals.

Optionally, each pulse oximeter includes at least a red, infrared, and green LED.

Optionally, one or both of the second electrode 106 and the second pulse oximeter 110 are integrated with a button of the wearable device 100.

Optionally, the first pulse oximeter 108 is configured to operate in a reflection mode.

Optionally, the housing 102 is formed to encircle at least a portion of the first arm and the first pulse oximeter 108 including two elements configured to operate in a transmission mode.

Optionally, the offset and/or one or more biometric lengths of the user are estimated using one or more parameters of the user and/or measured using a predefined calibration step.

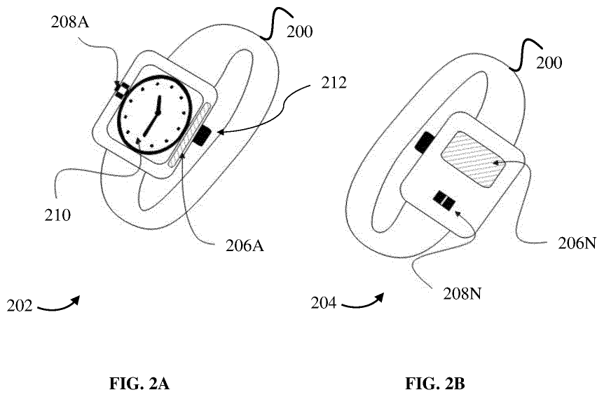

FIGS. 2A-2B illustrate exemplary diagrams of a front side 202 and a back side 204 of a wearable device 200 in accordance with an implementation of the disclosure. The wearable device 200 may be a smartwatch. Optionally, the wearable device 200 can be worn on hands of a user. Optionally, the wearable device 200 can be worn on arms, wrists, or fingers of the user. The wearable device 200 includes one or more electrodes 206A-N, one or more sensors 208A-N, a screen 210, and a button 212. The one or more electrodes 206A-N are configured to perform an electrocardiogram, ECG, measurement between two hands of the user. The one or more electrodes 206A-N includes a first electrode 206N and a second electrode 206A. When the wearable device 200 is worn by the user, the first electrode 206N is in contact with the wrists of the user and the second electrode 206A that is in contact with a finger of another hand, configured to perform the ECG measurement of the user.

The one or more sensors 208A-N includes a first sensor 208N and a second sensor 208A, that is configured to measure an oxygen saturation level of the user. When the first sensor 208N is in contact with the wrist of the user and the second sensor 208A is in contact with the user's finger of another hand, the one or more sensors 208A-N are configured to measure the oxygen saturation level of the user. The one or more sensors 208A-N may be known as a SPO2 sensor. The screen 210 is configured to display any of the ECG measurements or the oxygen saturation level measurement of the user. Optionally, the screen 210 displays an analog clock when the one or more electrodes 206A-N and the one or more sensors 208A-N are not in use. Optionally, the one or more electrodes 206A-N, the one or more sensors 208A-N, the screen 210, and the button 212 can be connected in a wristband, that can be worn by the user.

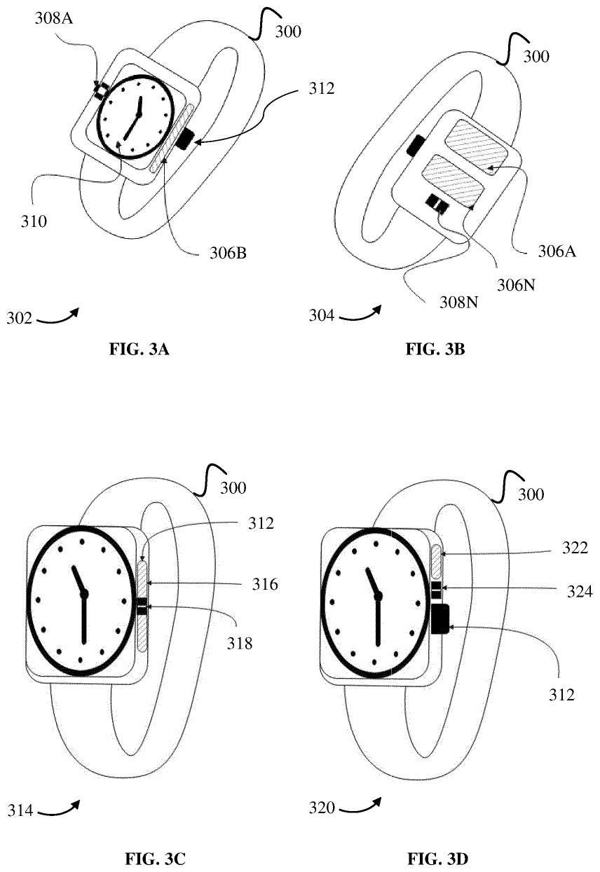

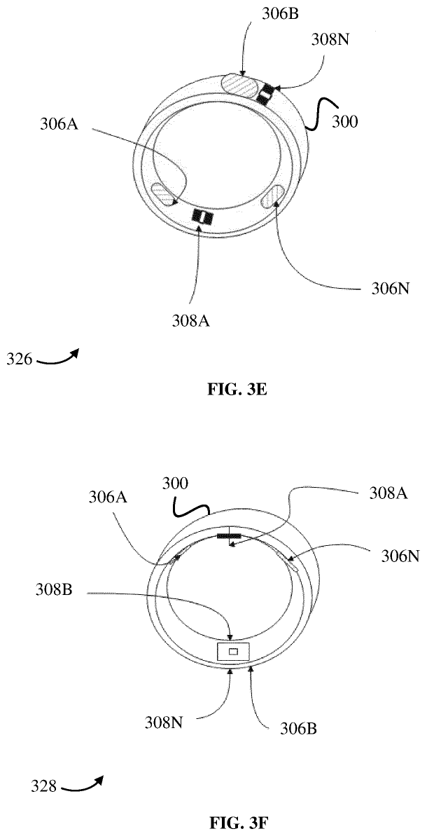

FIGS. 3A-3F illustrate exemplary diagrams of a wearable device 300 in accordance with an implementation of the disclosure. FIG. 3A and FIG. 3B illustrate exemplary diagrams of a front side 302 and a back side 304 of the wearable device 300. The wearable device 300 includes one or more electrodes 306A-N, one or more sensors 308A-N, a screen 310, and a button 312. The one or more electrodes 306A-N includes a first electrode 306A, a second electrode 306B, and a third electrode 306N. The third electrode 306N is configured to contact any of a part of arms, hands, or fingers of a user. The third electrode 306N is connected to ground which improves a quality of the electrocardiogram, ECG, signal measured between the first electrode 306A and the second electrode 306B.

FIG. 3C illustrates an exemplary diagram 314 of the wearable device 300 with a combined electrode and sensor. The exemplary diagram 314 of the wearable device 300 includes an ECG electrode 316, a SPO2 sensor 318, and the button 312. The ECG electrode 316 and the SPO2 sensor 318 are combined and included in the button 312 of the wearable device 300, which enables the user to contact any of the fingers or the opposite hand to activate the wearable device 300.

FIG. 3D illustrates an exemplary diagram 320 of the wearable device 300 with a separate electrode 322 and a sensor 324. The exemplary diagram 320 of the wearable device 300 includes the electrode 322, the sensor 324, and the button 312. The electrode 322 and the sensor 324 are placed separately from the button 312 and placed on a same side of the wearable device 300, which enables the user to contact both the electrode 322 and the sensor 324 with a same finger of the user. Optionally, the sensor 324 is a SPO2 sensor.

FIGS. 3E and 3F illustrate exemplary diagrams of the wearable device 300 in a top perspective view 326 and a bottom perspective view 328. Optionally, the wearable device 300 can be a ring that can be worn by the user on a finger. The wearable device 300 includes the one or more electrodes 306A-N, and the one or more sensors 308A-N. The one or more electrodes 306A-N includes the first electrode 306A, the second electrode 306B, and the third electrode 306N. The first electrode 306A and the third electrode 306N are in contact with the finger wearing the device 300, and the second electrode 306B is in contact with a finger of the opposite hand. The one or more sensors 308A-N includes a first sensor 308A, a second sensor 308B and a third sensor 308N. The first sensor 308A and the second sensor 308B are placed inside the wearable device 300 on opposite sides to enable a transmission mode. The first sensor 308A is in contact by the finger wearing the wearable device 300 and the second sensor 308B is to measure the SPO2 of the user in transmission mode. The first sensor 308A may emit light and the second sensor 308B may measure the transmitted light for the SPO2 measurement. The third sensor 308N is placed on the wearable device 300 that is contacted by the opposite hand of the user for SPO2 measurement in the reflection mode. Optionally, the one or more sensors 308A-N is a SPO2 sensor.

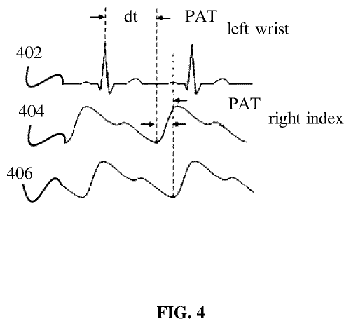

FIG. 4 depicts a graphical representation 400 of one or more signals acquired by a wearable device in accordance with an implementation of the disclosure. The wearable device is configured for the acquisition of the one or more signals from a user. The one or more signals includes an electrocardiogram, ECG signal 402, a photoplethysmogram, PPG, signal 404 of a left wrist of the user acquired by a first SPO2 sensor, and a photoplethysmogram, PPG, signal 406 of a right index of the user acquired by a second SPO2 sensor. Optionally, the wearable device can be configured for the acquisition of one or more PPG signals from the SPO2 sensors, which enables to improve the different timings calculation, for example, by selecting the one PPG signal with a best signal quality or combining the one or more PPG signals to obtain a more precise PPG signal.

The combination of the ECG signal 402, the PPG signal 404, and the PPG signal 406 of the user determines a pulse arrival time, PAT for a left arm and a right arm of the user. The one or more PPG signals may have a delay between the PPG signals as the PPG signal 404 and the PPG signal 406 are not acquired in a same location on each arms of the user. Optionally, a distance difference between the two locations can be approximated using any of a gender and a height of the user, or using an accelerometer of the wearable device, that enables the user to make a particular gesture to measure the distance during a calibration step. The delay may be measured also by performing mirrored measurements. In mirrored measurements, the wearable device is placed in turn to each hand and PATs are measured for both arms in both configurations. With the one or more signals, information obtained from the one or more signals are as follows:

$$\begin{matrix}{PAT}_{left wrist} = PEP + \frac{{L}_{aorta}}{{PWV}_{aorta}} + \frac{{L}_{arm}}{{PWV}_{left arm}} \\ {PAT}_{right index} = PEP + \frac{{L}_{aorta}}{{PWV}_{aorta}} + \frac{{L}_{arm}}{{PWV}_{right arm}} + \frac{{L}_{hand - index}}{{PWV}_{right arm}} \\ {TT}_{wrist - index} = {PAT}_{right index} - {PAT}_{left wrist} = \frac{{L}_{hand - index}}{{PWV}_{right arm}} + {L}_{arm} ( \frac{1}{{PWV}_{right arm}} - \frac{1}{{PWV}_{left arm}} )\end{matrix}$$

Optionally, in case of a symmetrical PWV between both the arms of the user, a pulse transit time, PTT, between the wrist and index is limited to the term, L_{hand-index}/PWV_{right arm}. Optionally, by knowing different lengths (L) and considering Pre-Ejection Period, PEP as a constant percentage of the PAT, a calibration in range can be found in case of symmetrical values of PWV between the arms so that α PAT_{left wrist}<PTT_{wrist-index}<β PAT_{left wrist} with α and β some constants determined previously while measuring on several people and dependent on a length of the arm of the user.

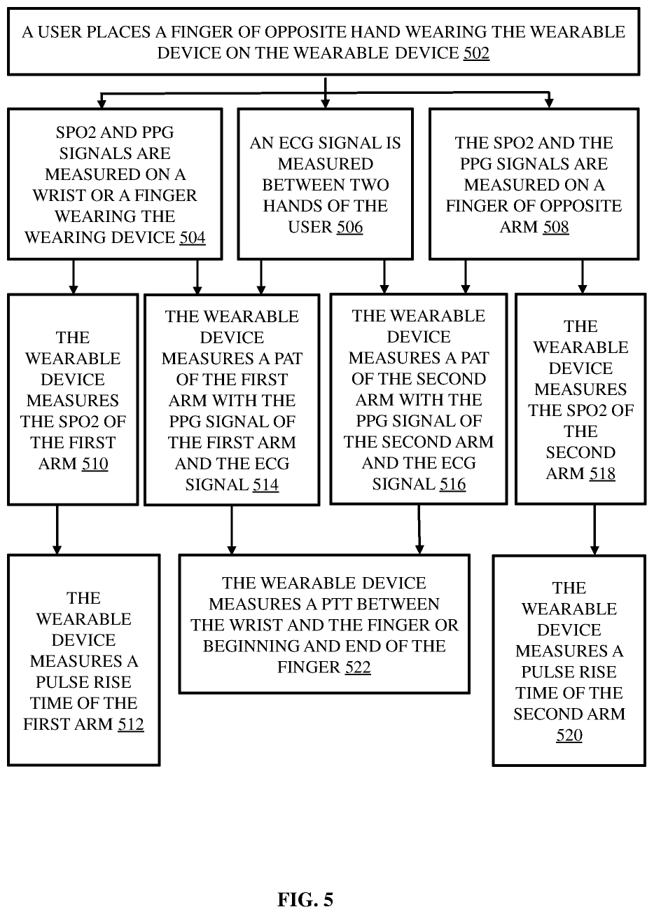

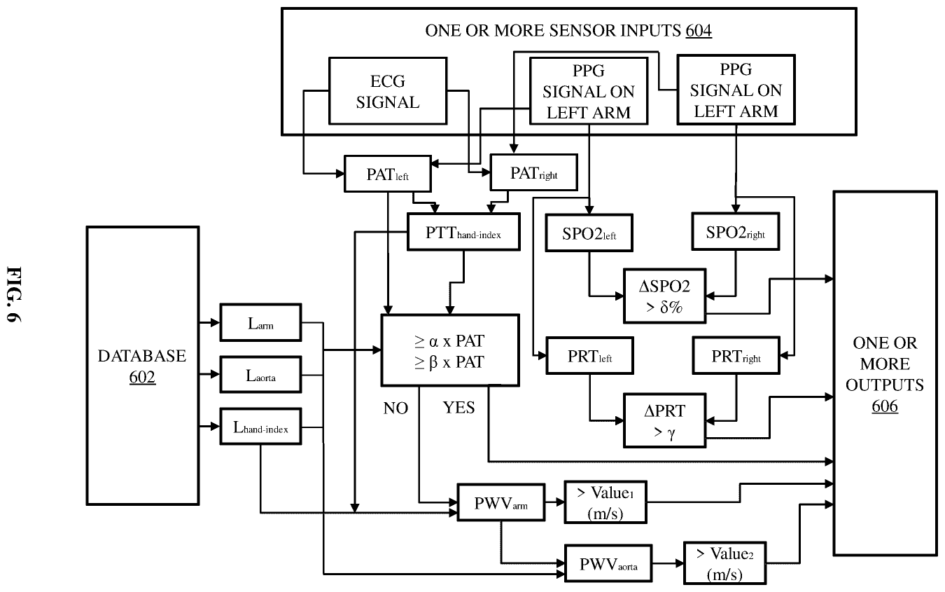

FIG. 5 illustrates a flow diagram of a sequence of measurement and acquisition of parameters in a wearable device in accordance with an implementation of the disclosure. At a step 502, a user places a finger of opposite hand wearing the wearable device on the wearable device.

Optionally, the wearable device is a smartwatch or a ring. At a step 504, SPO2 and PPG signals are measured on a wrist or a finger wearing the wearable device. At step 506, an ECG signal is measured between two hands of the user. At a step 508, the SPO2 and the PPG signals are measured on a finger of opposite arm. At a step 510, the wearable device measures the SPO2 of the first arm. At a step 512, the wearable device measures a pulse rise time of the first arm. At a step 514, the wearable device measures a PAT of the first arm with the PPG signal of the first arm and the ECG signal. At a step 516, the wearable device measures a PAT of the second arm with the PPG signal of the second arm and the ECG signal. At a step 518, the wearable device measures the SPO2 of the second arm. At a step 520, the wearable device measures a pulse rise time of the second arm. At a step 522, the wearable device measures a PTT between the wrist and the finger or beginning and end of the finger. The flow diagram enables a determination of different parameters that may be used in an algorithm to determine a cardiovascular risk of the user.

FIG. 6 illustrates a schematic diagram 600 of an algorithm to determine a cardiovascular risk of a user in accordance with an implementation of the disclosure. The schematic diagram 600 includes a database 602, one or more sensor inputs 604, and one or more outputs 606. Optionally, the algorithm is placed inside a wearable device to determine the cardiovascular risk of the user. The database 602 includes personal information of a user wearing the wearable device. The personal information of the user may include an age, a gender, or a height of the user. The wearable device enables the determination of the biological lengths, Lₐᵣₘ, Lₐₒᵣₜₐ, and L_{hand-index} based on the personal information of the user and/or a calibration step using the accelerometer of the device. The one or more sensor inputs 604 includes an ECG signal, a PPG signal on a left arm, and a PPG signal on a right arm. The one or more outputs 606 includes a risk of a narrowed artery, an asymmetrical PWV in the arms, and an increased cardiovascular risk.

The algorithm enables the wearable device to determine a pulse arrival time, PAT_{left} with the ECG signal, and the PPG signal on the left arm, a PAT_{right} with the ECG signal and the PPG signal on the right arm, a SPO2_{left} with the PPG signals on the left arm, and a SPO2_{right} with the PPG signals on the right arm. The algorithm determines a maximum acceptable SPO2 difference with the SPO2_{left} and the SPO2_{right}. The wearable device determines a PRT_{left} and a PRT_{right} with the PPG signal on the left arm and the PPG signal on the right arm. The algorithm determines a maximum acceptable pulse rise time difference with the PRT_{left} and the PRT_{right}. Optionally, the maximum acceptable pulse rise time difference can be pre-determined while developing the algorithm. The algorithm outputs the risk of the narrowed artery with the determined maximum acceptable SPO2 difference and the maximum acceptable pulse rise time difference.

The algorithm determines a pulse transit time, PTT_{hand-index} with the PAT_{left} and the PAT_{right} and a set of coefficients α and β dependent on Lₐᵣₘ and L_{hand-index} used to compare the PTT_{hand-index} to the PAT_{left} or the PAT_{right}. If the PTT_{hand-index} is not comprised in the proper range determined by α, β and PAT_{left} or PAT_{right}, the algorithm outputs that the pulse wave velocity, PWV, in the arms is asymmetrical. If it is not the case, the algorithm determines a PWVₐᵣₘ with PTT_{hand-index} and L_{hand-index} and compares it with a value in meter/second, and a PWVₐₒᵣₜₐ with PAT_{left}, PAT_{right}, PWVₐᵣₘ, Lₐᵣₘ, Lₐₒᵣₜₐ, and L_{hand-index} and compares it with a value₂ in meter/second, and outputs the increased cardiovascular risk. The value is a maximum acceptable PWV value for arms. Optionally, the maximum acceptable PWV value for arms depends on the age and gender of the user. The value₂ is a maximum acceptable PWV value for aorta. Optionally, the maximum acceptable PWV value for aorta depend on the age and gender of the user.

Optionally, the PWV is used to determine a stiffness of arteries and if a value is high for the age of the user, the algorithm outputs the increased cardiovascular risk. Optionally, the PWV is used to determine a blood pressure of the user. The algorithm may include one or more equations to obtain information on the cardiovascular risk of the user. The wearable device may include a machine learning algorithm combining one or more information to obtain more accurate values and determine the cardiovascular risk of the user. Optionally, the PPG signals including the PRT, a heart rate, features of pulse waveforms can be included to improve accuracy of the PWV determination. Optionally, the PPG signals obtained from one or more wavelengths are combined to obtain more accurate PWV determination.

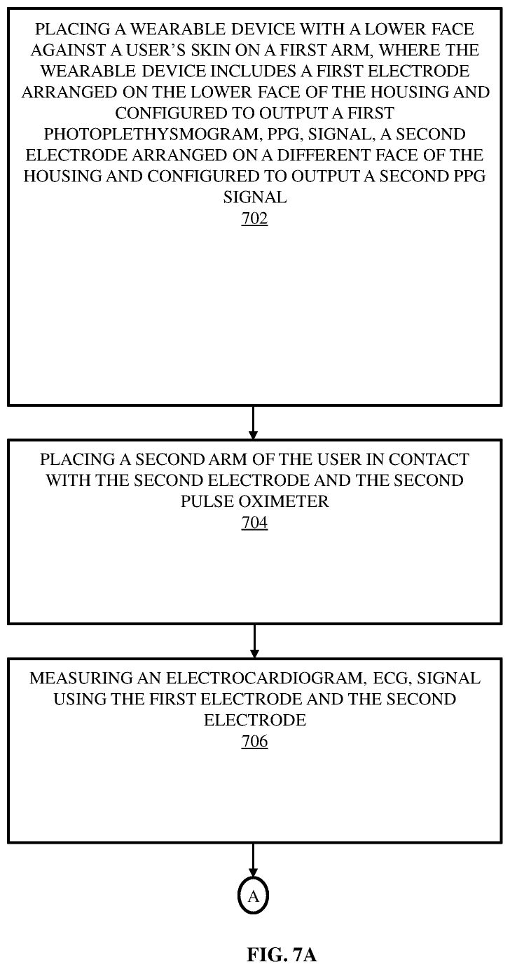

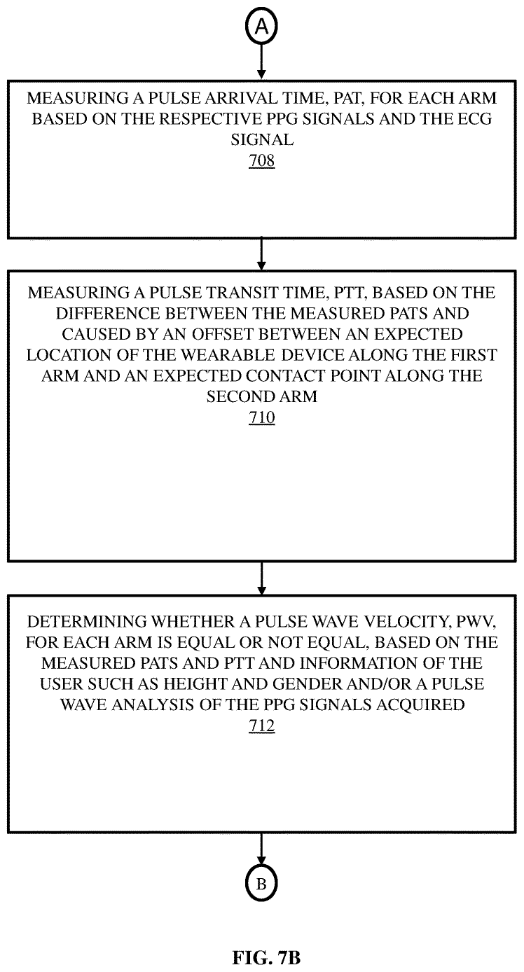

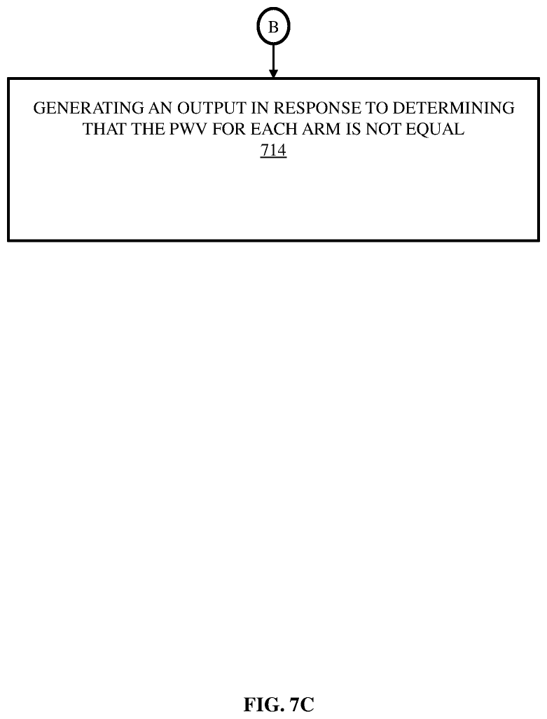

FIGS. 7A-7C are flow diagrams that illustrate a method of cardiovascular monitoring in accordance with an implementation of the disclosure. At a step 702, a wearable device is placed with a lower face against a user's skin on a first arm, where the wearable device includes a first electrode arranged on the lower face of the housing and configured to output a first photoplethysmogram, PPG, signal, a second electrode arranged on a different face of the housing and configured to output a second PPG signal. At a step 704, a second arm of the user is placed in contact with the second electrode and the second pulse oximeter. At a step 706, an electrocardiogram, ECG, signal is measured using the first electrode and the second electrode. At a step 708, a pulse arrival time, PAT, is measured for each arm based on the respective PPG signals and the ECG signal. At a step 710, a pulse transit time, PTT, is measured based on the difference between the measured PATs and caused by an offset between an expected location of the wearable device along the first arm and an expected contact point along the second arm.

At a step 712, whether a pulse wave velocity, PWV, for each arm is equal or not equal is determined, based on the measured PATs and PTT and information of the user such as height and gender and/or a pulse wave analysis of the PPG signals acquired. At a step 714, an output is generated in response to determining that the PWV for each arm is not equal.

The method provides calculation of a risk of having or developing a cardiovascular disease and in particular a Peripheral Artery Disease, PAD, or a Carotid Artery Disease, CAD. The method calculates the risk of having or developing a PAD or a CAD by evaluating the symmetry of parameters measured in both arms. The method includes a combination of measurements to obtain an indication of a cardiovascular health of the user and to improve a calculation of aortic pulse wave velocity, PWV. The method evaluates the cardiovascular health of the user by calculating peripheral PWV in the arms and including pulse arrival times, PATs to approximate an aortic PWV for the user with a symmetrical PWV in both arms. The method determines a blood pressure of the user using the aortic PWV.

Optionally, the method includes calculating, based on the first pulse oximeter and the second pulse oximeter signals, oxygen saturation values and pulse rise times, PRTs, for the first arm and the second arm. The method includes comparing the SPO2 values and PRTs for the first arm and the second arm to identify a potential obstruction, and generating an output in response to any potential obstruction.

Optionally, the method further includes if the PWV for each is equal, calculating a peripheral PWV based on the measured PATs and PTT, and generating an output in response to a value of the peripheral PWV above a predefined threshold.

Optionally, the method further includes calculating an aortic PWV from the calculated peripheral PWV and the measured PATs, and generating an output in response to a value of the aortic PWV outside a predefined range.

Optionally, the method further includes calculating a blood pressure of the user based on the calculated aortic PWV. Optionally, the calculation is calibrated using an external blood pressure measurement. The method further includes generating an output based on the calculated blood pressure.

Optionally, there is provided a computer-readable medium including instructions which, when executed by a processor, cause the processor to perform the method.

It should be understood that the arrangement of components illustrated in the figures described are exemplary and that other arrangement may be possible. It should also be understood that the various system components (and means) defined by the claims, described below, and illustrated in the various block diagrams represent components in some systems configured according to the subject matter disclosed herein. For example, one or more of these system components (and means) may be realized, in whole or in part, by at least some of the components illustrated in the arrangements illustrated in the described figures.

In addition, while at least one of these components are implemented at least partially as an electronic hardware component, and therefore constitutes a machine, the other components May be implemented in software that when included in an execution environment constitutes a machine, hardware, or a combination of software and hardware.

Although the disclosure and its advantages have been described in detail, it should be understood that various changes, substitutions, and alterations can be made herein without departing from the spirit and scope of the disclosure as defined by the appended claims.

1. A wearable device, comprising:
a housing comprising a lower face and a first face and configured to be placed with the lower face placed in contact with a first arm of a user;
a first electrode arranged on the lower face;
a second electrode arranged on the first face;
a first pulse oximeter arranged on the lower face and configured to output a first photoplethysmogram, (PPG) signal when the lower face is placed in contact with the first arm;
a second pulse oximeter arranged on the first face and configured to output a second PPG signal when the second electrode and the second pulse oximeter are in contact with a second arm of the user;
a processor coupled to the first electrode and the second electrode and configured to:
measure an electrocardiogram (ECG) signal using the first electrode and the second electrode when the housing is placed with the lower face in contact with the first arm and the second electrode is in contact with the second arm;
measure a first pulse arrival time, (PAT) for the first arm based on the first PPG signal and the ECG signal;
measure a second PAT for the second arm based on the second PPG signal and the ECG signal;
measure a pulse transit time, (PTT) based on a difference between the first PAT and the second PAT; and
determine whether a first pulse wave velocity (PWV) for the first arm and a second PWV for the second arm are equal or not equal based on the first PAT, the second PAT, the PTT, and an offset between an expected location of the wearable device along the first arm and an expected contact point along the second arm or a pulse waveform analysis of the first PPG signal and the second PPG signal; and
an output system configured to generate a first output when the first PWV and the second PWV are not equal.

2. The wearable device of claim 1, wherein the processor is further configured to:
calculate a first oxygen saturation (SPO2) value and a first pulse rise time (PRT) for the first arm based on a first combination of a plurality of third PPG signals from the first pulse oximeter on the first arm;
calculate a second SPO2 value and a second PRT for the second arm based on a second combination of a plurality of fourth PPG signals from the second pulse oximeter on the second arm; and
compare the first SPO2 value and the second SPO2 value and compare the first PRT and the second PRT to identify a potential obstruction, and
wherein the output system is further configured to generate a second output in response to the potential obstruction.

3. The wearable device of claim 1, wherein, when the first PWV and the second PWV are equal, the processor is further configured to calculate a peripheral PWV based on the first PAT, the second PAT, and the PTT, and wherein the output system is further configured to generate a second output in response to a first value of the peripheral PWV being above a predefined threshold.

4. The wearable device of claim 3, wherein the processor is further configured to calculate an aortic PWV from the peripheral PWV, the first PAT, and the second PAT, and wherein the output system is further configured to generate a third output in response to a second value of the aortic PWV being outside a predefined range.

5. The wearable device of claim 4, wherein the processor is further configured to calculate a blood pressure of the user based on the aortic PWV and using an external blood pressure measurement, and wherein the output system is further configured to generate a fourth output based on the blood pressure.

6. The wearable device of claim 1, wherein the output system includes a screen, a speaker, and/or a transmitter.

7. The wearable device of claim 1, further comprising a third electrode arranged on the lower face and coupled to a ground, wherein the processor is further configured to measure the ECG signal additionally using the third electrode.

8. The wearable device of claim 1, wherein each of the first pulse oximeter and the second pulse oximeter is further configured to use a plurality of wavelengths to generate a corresponding number of third PPG signals.

9. The wearable device of claim 8, wherein each of the first pulse oximeter and the second pulse oximeter comprises a red light-emitting diode (LED), an infrared LED, and a green LED.

10. The wearable device of claim 1, further comprising a button, wherein the second electrode or the second pulse oximeter is integrated with the button.

11. The wearable device of claim 1, wherein the first pulse oximeter is configured to operate in a reflection mode.

12. The wearable device of claim 1, wherein the housing is further configured to encircle a portion of the first arm, and wherein the first pulse oximeter comprises two elements configured to operate in a transmission mode.

13. The wearable device of claim 1, wherein the processor is further configured to:
estimate the offset or one or more biometric lengths of the user using one or more parameters of the user; or
measure the offset or the one or more biometric lengths using a predefined calibration step.

14. A method comprising:
placing a lower face of a housing of a wearable device in contact with a first arm of a user;
placing a second electrode of the wearable device and a second pulse oximeter of the wearable device in contact with a second arm of the user;
measuring an electrocardiogram (ECG) signal using a first electrode of the wearable device and the second electrode;
measuring a first pulse arrival time (PAT) for the first arm based on a first photoplethysmogram (PPG) signal and the ECG signal;
measuring a second PAT for the second arm based on a second PPG signal and the ECG signal;
measuring a pulse transit time (PTT) based on a difference between the first PAT and the second PAT;
determining whether a first pulse wave velocity (PWV) for the first arm and a second PWV for the second arm are equal or not equal based on the first PAT, the second PAT, the PTT, and an offset between an expected location of the wearable device along the first arm and an expected contact point along the second arm or a pulse wave analysis of the first PPG signal and the second PPG signal; and
generating a first output in response to determining that the first PWV and the second PWV are not equal.

15. The method of claim 14, further comprising:
calculating a first oxygen saturation (SPO2) value and a first pulse rise time (PRT) for the first arm based on a first combination of a plurality of third PPG signals from a first pulse oximeter on the first arm;
calculating a second SPO2 value and a second PRT for the second arm based on the second pulse oximeter;
comparing the first SPO2 value and the second SPO2 value and comparing the first PRT and the second PRT to identify a potential obstruction; and
generating a second output in response to the potential obstruction.

16. The method of claim 14, wherein when the first PWV and the second PWV are equal, the method further comprises:
calculating a peripheral PWV based on the first PAT, the second PAT, and the PTT; and
generating a second output in response to a first value of the peripheral PWV being above a predefined threshold.

17. The method of claim 16, further comprising:
calculating an aortic PWV from the peripheral PWV, the first PAT, and the second PAT; and
generating a third output in response to a second value of the aortic PWV being outside a predefined range.

18. The method of claim 17, further comprising:
calculating a blood pressure of the user based on the aortic PWV and using an external blood pressure measurement; and
generating a fourth output based on the blood pressure.

19. A computer program product comprising computer-executable instructions that are stored on a computer-readable medium and that, when executed by a processor, cause a wearable device to:
measure an electrocardiogram (ECG) signal using a first electrode of the wearable device and a second electrode of the wearable device based on a lower face of a housing of the wearable device is being placed in contact with a first arm of a user and based on the second electrode and a second pulse oximeter of the wearable device are being placed in contact with a second arm of the user, wherein the first electrode is arranged on the lower face, wherein the second electrode is arranged on a first face of the housing, and wherein the second pulse oximeter is arranged on the first face and configured to output a second photoplethysmogram (PPG) signal;
measure a first pulse arrival time (PAT) for the first arm based on a first PPG signal and the ECG signal, wherein the wearable device further comprises a first pulse oximeter arranged on the lower face and configured to output the first PPG signal;
measure a second PAT for the second arm based on the second PPG signal and the ECG signal;
measure a pulse transit time (PTT) based on a difference between the first PAT and the second PAT;
determine whether a first pulse wave velocity (PWV) for the first arm and a second PWV for the second arm are equal or not equal based on the first PAT, the second PAT, the PTT, and an offset between an expected location of the wearable device along the first arm and an expected contact point along the second arm or a pulse wave analysis of the first PPG signal and the second PPG signal; and
generate a first output when the first PWV and the second PWV are not equal.

20. The computer program product of claim 19, wherein when the first PWV and the second PWV are equal and when executed by the processor, the computer-executable instructions further cause the wearable device to:
calculate a peripheral PWV based on the first PWV, the second PWV, and the PTT; and
generate a second output in response to a value of the peripheral PWV being above a predefined threshold.
